(19) United States
(12) Patent Application Publication
(10) Pub. No.: US 2021/0049742 A1
(43) Pub. Date: Feb. 18, 2021

(54) IMAGE PROCESSING APPARATUS, IMAGE PROCESSING METHOD, AND NON-TRANSITORY COMPUTER-READABLE STORAGE MEDIUM

(71) Applicant: CANON KABUSHIKI KAISHA, Tokyo (JP)

(72) Inventors: Marek Rozanski, Wroclaw (PL); Yukio Sakagawa, Tokyo (JP)

(21) Appl. No.: 17/073,031

(22) Filed: Oct. 16, 2020

Related U.S. Application Data
(63) Continuation of application No. PCT/JP2019/015661, filed on Apr. 10, 2019.

(30) Foreign Application Priority Data
Apr. 19, 2018 (JP) 2018-080765

(51) Int. Cl. G06T 5/00; A61B 3/10; A61B 5/00; G06T 7/00; G06T 3/40; G06T 7/13

(52) U.S. Cl. CPC G06T 5/002; A61B 3/102; A61B 5/489; G06T 7/0012; G06T 3/40; G06T 2207/20092; G06T 2207/10101; G06T 2207/30041; G06T 2207/30101; G06T 2207/30168; G06T 7/13

(57) ABSTRACT

An image processing apparatus for reducing a projection artifact in motion contrast data of a subject's eye includes a calculation unit configured to calculate, using information on a position of a blood vessel structure of the subject's eye and OCT intensity information on the subject's eye, an attenuation coefficient regarding attenuation of the motion contrast data in a direction of depth of the subject's eye, and a correction unit configured to execute correction processing on the motion contrast data using the calculated attenuation coefficient.

### CROSS-REFERENCE TO RELATED APPLICATIONS

This application is a Continuation of International Patent Application No. PCT/JP2019/015661, filed Apr. 10, 2019, which claims the benefit of Japanese Patent Application No. 2018-080765, filed Apr. 19, 2018, both of which are hereby incorporated by reference herein in their entirety.

### TECHNICAL FIELD

The disclosed technology relates to an image processing apparatus, an image processing method, and a non-transitory computer-readable storage medium.

### BACKGROUND ART

With the use of an ophthalmic tomographic imaging device such as an optical coherence tomography (OCT) device, the state of the inside of the retinal layer can be three-dimensionally observed. Such a tomographic imaging device is widely used to conduct an ophthalmological examination because the tomographic imaging device is useful for making a diagnosis of a disease more accurately. A form of an OCT device is, for example, a time domain OCT (TD-OCT) device, which is obtained by combining a broad-band light source and a Michelson interferometer. This is configured to measure interference light regarding back-scattered light obtained through a signal arm by moving the position of a reference mirror at a constant speed and obtain a reflected light intensity distribution in the direction of depth. However, it is difficult to acquire images at high speed because mechanical scanning needs to be performed with such a TD-OCT device. Thus, spectral domain OCT (SD-OCT), in which a broad-band light source is used and a spectrometer obtains a coherent signal, and swept source OCT (SS-OCT), in which light is temporally analyzed using a high-speed swept light source, have been developed as higher-speed image acquisition methods, so that a wider angle tomographic image can be acquired.

In contrast, fundus fluorescein angiography examination, which is invasive, has been performed so far to determine the state of a disease of fundus blood vessels when an ophthalmological examination is conducted. In recent years, OCT angiography (hereinafter referred to as OCTA) techniques have been used with which fundus blood vessels are noninvasively three-dimensionally represented using OCT. In OCTA, the same position is scanned a plurality of times with measurement light, and motion contrast caused by the interaction between displacement of red blood cells and the measurement light is converted into an image. FIG. 4 illustrates an example of OCTA imaging, in which a main scanning direction is the horizontal (the x axis) direction and a B-scan is consecutively performed r times at individual positions (yi; 1≤i≤n) in a sub-scanning direction (the y axis direction). Note that, in OCTA imaging, scanning of the same position a plurality of times is called cluster scanning, and a plurality of tomographic images obtained at the same position are called a cluster. Motion contrast data is generated for each cluster, and the contrast of an OCTA image is known to be improved when the number of tomographic images per cluster (the number of times of scanning of substantially the same position) is increased.

In this case, a projection artifact is known, which is a phenomenon in which the motion contrast in a superficial retinal blood vessel is reflected on a deep layer side (a deep layer of the retina, the outer layer of the retina, the choroid coat), and a high decorrelation value occurs in a region on the deep layer side where no blood vessels are actually present. NPL 1 discloses that the step-down exponential filtering method reduces a projection artifact in motion contrast data. This is a method in which a projection artifact in motion contrast data is reduced by correcting the motion contrast data using an attenuation coefficient.

### CITATION LIST

### Non Patent Literature

NPL 1 Mahmud et al., “Review of speckle and phase variance optical coherence tomography to visualize microvascular networks”, Journal of Biomedical Optics 18 (5), 050901 (May, 2013)

### SUMMARY OF INVENTION

One of image processing apparatuses disclosed herein is an image processing apparatus for reducing a projection artifact in motion contrast data of a subject's eye, the image processing apparatus including a calculation unit configured to calculate, using information on a position of a blood vessel structure of the subject's eye and OCT intensity information on the subject's eye, an attenuation coefficient regarding attenuation of the motion contrast data in a direction of depth of the subject's eye, and a correction unit configured to execute correction processing on the motion contrast data using the calculated attenuation coefficient.

Further features of the present invention will become apparent from the following description of exemplary embodiments with reference to the attached drawings.

### BRIEF DESCRIPTION OF DRAWINGS

FIG. 1 is a block diagram illustrating the configuration of an image processing apparatus according to a first embodiment.

FIG. 2A is a diagram for describing an image processing system according to the first embodiment and an optical measurement system included in a tomographic imaging device included in the image processing system.

FIG. 2B is a diagram for describing the image processing system according to the first embodiment and the optical measurement system included in the tomographic imaging device included in the image processing system.

FIG. 3A is a flow chart of processing executable by the image processing system according to the first embodiment.

FIG. 3B is a flow chart of processing executable by the image processing system according to the first embodiment.

FIG. 4 is a diagram for describing a scan method for OCTA imaging in the first embodiment.

FIG. 5A is a diagram for describing processing executed in S320 of the first embodiment.

FIG. 5B is a diagram for describing processing executed in S320 of the first embodiment.

FIG. 6A is a diagram for describing processing executed in S330 of the first embodiment.

FIG. 6B is a diagram for describing processing executed in S330 of the first embodiment.

FIGS. 7A and 7B are diagrams for describing processing executed in S340 of the first embodiment.

FIG. 8 is a diagram illustrating an example of processing results of the first embodiment.

FIG. 9A is a flow chart of processing executable by an image processing system according to a second embodiment.

FIG. 9B is a flow chart of processing executable by the image processing system according to the second embodiment.

FIG. 10A is a diagram for describing processing executed in S920 of a third embodiment.

FIG. 10B is a diagram for describing processing executed in S920 of the third embodiment.

### DESCRIPTION OF EMBODIMENTS

When a fixed attenuation coefficient is used as before, there is a limit to the extent to which a projection artifact is reduced in motion contrast data. For example, even when an attenuation coefficient is adjusted such that a projection artifact can be reduced in a layer at a predetermined depth, a projection artifact in a layer at another depth may not be sufficiently reduced. In a present embodiment, a projection artifact is effectively reduced in motion contrast data.

In addition to this, the individual configurations of embodiments to be described later make it possible to provide operational effects that cannot be achieved by existing technologies.

### First Embodiment

One of image processing apparatuses according to the present embodiment includes a calculation unit that uses information on the position of a blood vessel structure such as a large vessel structure (LVS) of the subject's eye and OCT intensity information on the subject's eye to calculate an attenuation coefficient regarding attenuation of motion contrast data in the direction of depth of the subject's eye. In addition, the one of the image processing apparatuses according to the present embodiment includes a correction unit that executes, using the attenuation coefficient, correction processing on the motion contrast data. For example, the calculation unit calculates the attenuation coefficient using OCT intensity information on the position of a portion deeper than the blood vessel structure. As a result, an actual degree of effect of a projection artifact caused by the blood vessel structure can be reflected on the attenuation coefficient. Thus, for example, the correction processing can be prevented from being executed too severely or too lightly. That is, a projection artifact in motion contrast data can be effectively reduced. In this case, the information on the position may be any information that enables the position to be recognized. The information on the position may be, for example, a coordinate value in the direction of depth of the subject's eye (the Z direction) or may also be three-dimensional coordinate values. In addition, the information on the position is, for example, information on the distance from the blood vessel structure in the direction of depth of the subject's eye. In this case, the information on the distance may be any information that enables the distance to be recognized. The information on the distance may be, for example, a numerical value with units or may also be something that can eventually lead to the distance such as two coordinate values.

In addition, the one of the image processing apparatuses according to the present embodiment includes a determination unit that determines, using information on a comparison result between OCT intensity information on the inside of the blood vessel structure of the subject's eye and OCT intensity information on the outside of the blood vessel structure, whether to execute the correction processing for the blood vessel structure. For example, in a case where the OCT intensity information on the outside of the blood vessel structure is lower than the OCT intensity information on the inside of the blood vessel structure, the determination unit determines that the correction processing is to be executed for the blood vessel structure. Depending on a blood vessel structure, no projection artifact may occur. When existing correction processing is performed for such a blood vessel structure as in the case where a projection artifact has occurred, an erroneous image may be generated. Thus, whether a projection artifact has occurred can be checked through a determination made by the determination unit described above. This can effectively reduce a projection artifact in motion contrast data. In this case, the information on the comparison result may be any information that enables the comparison result to be recognized. Note that, regarding a plurality of blood vessel structures of the subject's eye, whether to execute the correction processing may be determined for each blood vessel structure. Consequently, it is possible to check whether a projection artifact has occurred for each of the plurality of blood vessel structures. In the following, an image processing system including an image processing apparatus according to the present embodiment will be described with reference to the drawings.

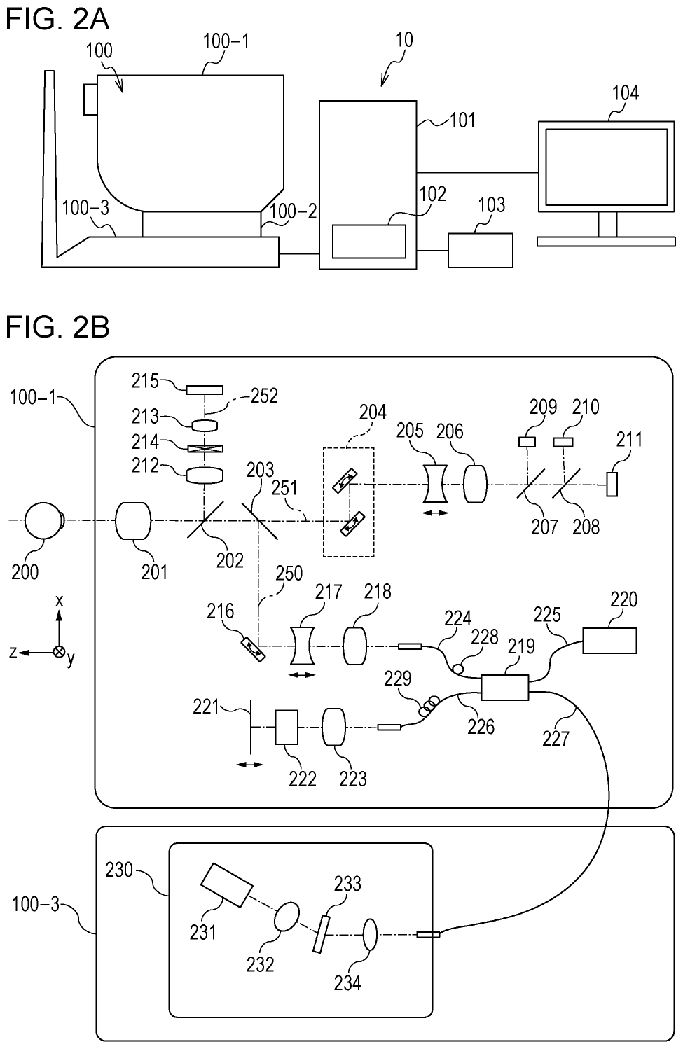

FIGS. 2A and 2B are diagrams illustrating the configuration of an image processing system 10 including an image processing apparatus 101 according to the present embodiment. As illustrated in FIG. 2A, the image processing system 10 is formed by connecting the image processing apparatus 101 to a tomographic imaging device 100 (also called an OCT device), an external storage unit 102, an input unit 103, and a display unit 104 via an interface.

The tomographic imaging device 100 is a device that captures ophthalmic OCT images. In the present embodiment, an SD-OCT device is used as the tomographic imaging device 100. Instead of the SD-OCT device, for example, an SS-OCT device may be used as the tomographic imaging device 100.

In FIG. 2A, an optical measurement system 100-1 is an optical system for acquiring an anterior eye segment image, an SLO fundus image of the subject's eye, and a tomographic image. A stage unit 100-2 enables the optical measurement system 100-1 to move right and left and backward and forward. A base unit 100-3 includes a spectrometer, which will be described later. The image processing apparatus 101 is a computer that, for example, controls the stage unit 101-2, controls an alignment operation, and reconstructs tomographic images. The external storage unit 102 stores, for example, programs for capturing tomographic images, patient information, image capturing data, and image data and measurement data regarding examinations conducted in the past. The input unit 103 sends a command to the computer, and specifically includes a keyboard and a mouse. The display unit 104 includes, for example, a monitor.

Configuration of Tomographic Imaging Device

The configuration of an optical measurement system and a spectrometer in the tomographic imaging device 100 in the present embodiment will be described using FIG. 2B. First, the inside of the optical measurement system 100-1 will be described. An objective lens 201 is placed so as to face a subject's eyes 200, and a first dichroic mirror 202 and a second dichroic mirror 203 are arranged on the optical axis of the objective lens 201. These dichroic mirrors divide light into an optical path 250 for an OCT optical system, an optical path 251 for an SLO optical system and a fixation lamp, and an optical path 252 for anterior eye observation on a wavelength band basis.

The optical path 251 for the SLO optical system and the fixation lamp has an SLO scanning unit 204, lenses 205 and 206, a mirror 207, a third dichroic mirror 208, an avalanche photodiode (APD) 209, an SLO light source 210, and a fixation lamp 211. The mirror 207 is a prism on which a perforated mirror or a hollow mirror has been vapor-deposited, and separates illumination light from the SLO light source 210 and light returning from the subject's eye. The third dichroic mirror 208 splits light into the optical path for the SLO light source 210 and the optical path for the fixation lamp 211 on a wavelength band basis. The SLO scanning unit 204 scans light emitted from the SLO light source 210 across the subject's eyes 200, and includes an X scanner, which scans in the X direction, and a Y scanner, which scans in the Y direction. In the present embodiment, the X scanner includes a polygon mirror because high-speed scan needs to be performed, and the Y scanner includes a galvanometer mirror. The lens 205 is driven by an unillustrated motor in order to achieve focusing for the SLO optical system and the fixation lamp 211. The SLO light source 210 generates light having a wavelength of about 780 nm. The APD 209 detects light returning from the subject's eye. The fixation lamp 211 emits visible light and leads the subject to fixate his or her eyes. Light emitted from the SLO light source 210 is reflected by the third dichroic mirror 208, passes through the mirror 207 and the lenses 206 and 205, and is scanned on the subject's eyes 200 by the SLO scanning unit 204. After retracing the same path as illumination light, light returning from the subject's eye 200 is reflected by the mirror 207 and is led to the APD 209, and then an SLO fundus image is obtained. Light emitted from the fixation lamp 211 passes through the third dichroic mirror 208 and the mirror 207, passes through the lenses 206 and 205, is formed into a predetermined shape at an arbitrary position on the subject's eyes 200 by the SLO scanning unit 204, and leads the subject to fixate his or her eyes.

In the optical path 252 for anterior eye observation, lenses 212 and 213, a split prism 214, and a charge-coupled device (CCD) 215 for anterior eye observation are arranged, the CCD 215 detecting infrared light. The CCD 215 is sensitive to wavelengths of unillustrated illumination light for anterior eye observation, specifically wavelengths of about 970 nm. The split prism 214 is arranged at a conjugate position with respect to the pupil of the subject's eyes 200 and can detect the distance of the optical measurement system 100-1 with respect to the subject's eyes 200 in the Z axis direction (the optical axis direction) as a split image of the anterior eye segment.

The optical path 250 for the OCT optical system is included in the OCT optical system as described above, and is used to capture a tomographic image of the subject's eye 200. More specifically, the optical path 250 is used to obtain a coherent signal for forming a tomographic image. An XY scanner 216 is used to scan light across the subject's eyes 200, and is illustrated as a mirror in FIG. 2B; however, the XY scanner 216 is actually a galvanometer mirror that performs scanning in both of the X axis direction and the Y axis direction. Among lenses 217 and 218, the lens 217 is driven by an unillustrated motor to focus, on the subject's eyes 200, light emitted from an OCT light source 220 and exiting from a fiber 224 connected to an optical coupler 219. By this focusing, light returning from the subject's eyes 200 is simultaneously formed into an image in a spot-like manner at and enter a leading end of the fiber 224. Next, the configuration of an optical path from the OCT light source 220, a reference optical system, and a spectrometer will be described. Reference number 220 denotes an OCT light source, 221 a reference mirror, 222 a dispersion compensation glass element, 223 a lens, 219 an optical coupler, 224 to 227 single-mode optical fibers integrally connected to the optical coupler, and 230 a spectrometer. These elements constitute a Michelson interferometer. Light emitted from the OCT light source 220 passes through the optical fiber 225 and is divided into measurement light for the optical fiber 224 and reference light for the optical fiber 226 via the optical coupler 219. The measurement light passes through the optical path for the OCT optical system described above, is caused to illuminate the subject's eye 200, which is an observation target, and reaches the optical coupler 219 through the same optical path by being reflected and scattered by the subject's eye 200.

In contrast, the reference light reaches the reference mirror 221 via the optical fiber 226, the lens 223, and the dispersion compensation glass element 222, which is inserted to achieve chromatic dispersion for the measurement light and reference light, and is then reflected by the reference mirror 221. Reflected light retraces the same optical path, and reaches the optical coupler 219. The measurement light and reference light are multiplexed by the optical coupler 219 and become interference light. In this case, interference occurs when the optical path for the measurement light and the optical path for the reference light are of substantially the same length. The reference mirror 221 is held in an adjustable manner in the optical axis direction by an unillustrated motor and an unillustrated driving mechanism, and is capable of matching the length of the optical path for the reference light to the length of the optical path for the measurement light. Interference light is led to the spectrometer 230 via the optical fiber 227. In addition, polarization adjusting units 228 and 229 are respectively provided in the optical fibers 224 and 226, and perform polarization adjustment. These polarization adjusting units have some portions formed by routing the optical fibers in a loop-like manner. For each of these loop-like portions, the fiber is twisted by rotating the loop-like portion on an axis corresponding to the longitudinal direction of the fiber, and a polarization state of the measurement light and that of the reference light can be individually adjusted and matched. The spectrometer 230 includes lenses 232 and 234, a diffraction grating 233, and a line sensor 231. Interference light exiting from the optical fiber 227 becomes parallel light via the lens 234. The parallel light is then analyzed by the diffraction grating 233 and is formed into an image on the line sensor 231 by the lens 232.

Next, the OCT light source 220 will be described. The OCT light source 220 is a super luminescent diode (SLD), which is a typical low coherent light source. The center wavelength is 855 nm, and the wavelength bandwidth is about 100 nm. In this case, the bandwidth is an important parameter because the bandwidth affects the optical-axis-direction resolution of a tomographic image to be captured. In this case, an SLD is selected as the type of light source; however, any light source that can emit low coherent light is acceptable, and for example an amplified spontaneous emission (ASE) source may be used. Considering that eye measurement is performed, a wavelength of near infrared light is appropriate as the center wavelength. In addition, the center wavelength affects the lateral resolution of a tomographic image to be captured, and thus preferably the center wavelength is as short as possible. Based on these two reasons, the center wavelength is set to 855 nm.

In the present embodiment, a Michelson interferometer is used as the interferometer; however, a Mach-Zehnder interferometer may be used. In accordance with the difference between the light intensity of the measurement light and that of the reference light, a Mach-Zehnder interferometer is preferably used in a case where the difference in light intensity is large, and a Michelson interferometer is preferably used in a case where the difference in light intensity is relatively small.

Configuration of Image Processing Apparatus

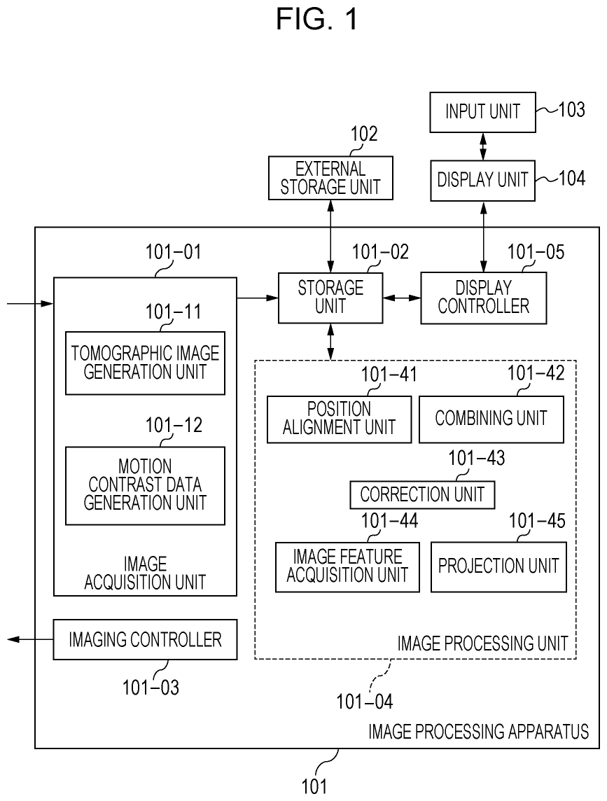

The configuration of the image processing apparatus 101 in the present embodiment will be described using FIG. 1. The image processing apparatus 101 is a personal computer (PC) connected to the tomographic imaging device 100, and includes an image acquisition unit 101-01, a storage unit 101-02, an imaging controller 101-03, an image processing unit 101-04, and a display controller 101-05. In addition, the function of the image processing apparatus 101 is realized by a central processing unit (CPU) executing software modules that realize the image acquisition unit 101-01, the imaging controller 101-03, the image processing unit 101-04, and the display controller 101-05. The present invention is not limited to this configuration. For example, the image processing unit 101-04 may be realized by a special-purpose hardware device such as an application-specific integrated circuit (ASIC), and the display controller 101-05 may be realized using a special-purpose processor such as a graphics processing unit (GPU), which is different from a CPU. In addition, the tomographic imaging device 100 may be connected to the image processing apparatus 101 via a network.

The image acquisition unit 101-01 acquires signal data of an SLO fundus image and a tomographic image captured by the tomographic imaging device 100. The image acquisition unit 101-01 has a tomographic image generation unit 101-11 and a motion contrast data generation unit 101-12. The tomographic image generation unit 101-11 acquires signal data (a coherent signal) of a tomographic image captured by the tomographic imaging device 100, generates a tomographic image by performing signal processing, and stores the generated tomographic image in the storage unit 101-02. The imaging controller 101-03 performs imaging control on the tomographic imaging device 100. The imaging control includes issue of commands to the tomographic imaging device 100 such as a command regarding setting of imaging parameters and a command regarding start or end of imaging.

The image processing unit 101-04 has a position alignment unit 101-41, a combining unit 101-42, a correction unit 101-43, an image feature acquisition unit 101-44, and a projection unit 101-45. The image acquisition unit 101-01 and the combining unit 101-42 described above are an example of an acquisition unit according to the present invention. The combining unit 101-42 combines, on the basis of a position alignment parameter obtained by the position alignment unit 101-41, a plurality of pieces of motion contrast data generated by the motion contrast data generation unit 101-12, and generates a combined motion contrast image. The correction unit 101-43 performs processing in which a projection artifact occurring in a motion contrast image is two-dimensionally or three-dimensionally reduced. The image feature acquisition unit 101-44 acquires the position of the layer boundary between the retina and the choroid coat, the position of the fovea centralis, and the position of the center of the optic disc from a tomographic image. The projection unit 101-45 projects a motion contrast image having a depth range based on the position of the layer boundary acquired by the image feature acquisition unit 101-44, and generates a motion contrast en-face image. The external storage unit 102 associates, with each other, and stores information on the subject's eye (a patient's name, age, gender, and so on), a captured image (a tomographic image, an SLO image, or an OCTA image), a combined image, an imaging parameter, position data on a blood vessel region and position data on a blood vessel center line, a measurement value, and a parameter set by the operator. The input unit 103 includes, for example, a mouse, a keyboard, and a touch operation screen. The operator sends commands to the image processing apparatus 101 and the tomographic imaging device 100 via the input unit 103. Note that as the configuration of the image processing apparatus 101 in the present invention, all the structural elements described above are not necessary, and for example the position alignment unit 101-41, the combining unit 101-42, and the projection unit 101-45 may be omitted.

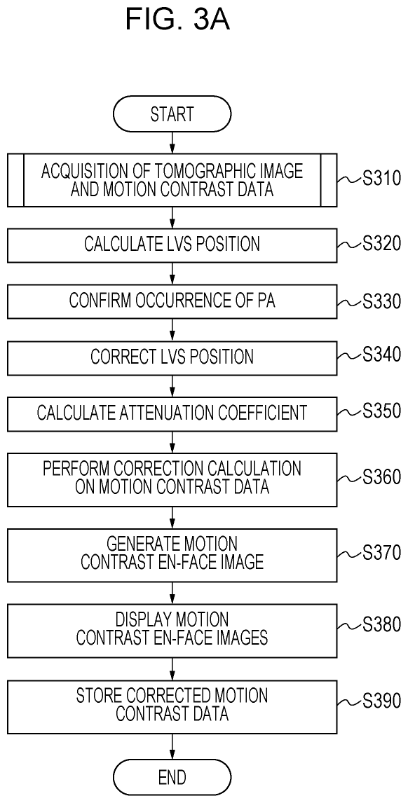

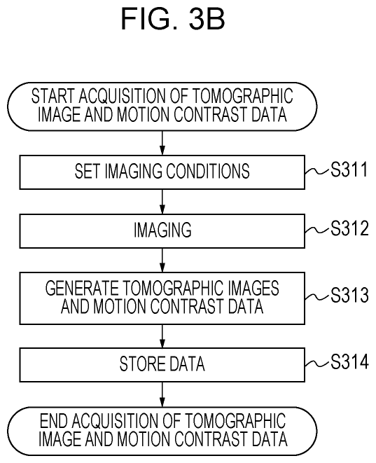

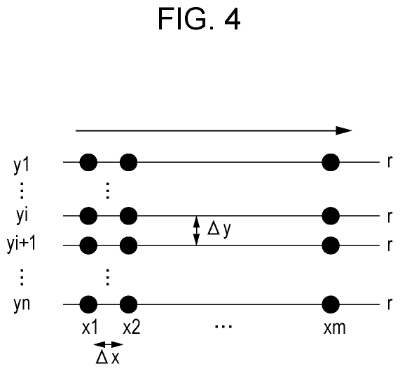

Next, processing steps of the image processing apparatus 101 of the present embodiment will be described with reference to FIG. 3A. FIG. 3A is a flow chart illustrating operation processing of the entire system in the present embodiment. Note that, in the present invention, generation of a motion contrast en-face image and so on in step S370 is an inessential processing step, and thus the processing step may be omitted.

Step S310

In step S310, the image processing unit 101-04 acquires an OCT tomographic image and motion contrast data. The image processing unit 101-04 may acquire an OCT tomographic image and motion contrast data that have already been stored in the external storage unit 102; however, the present embodiment describes an example in which an OCT tomographic image and motion contrast data are acquired by controlling the optical measurement system 100-1. Details of these processes will be described later. In the present embodiment, the way in which an OCT tomographic image and motion contrast data are acquired is not limited to this acquisition method. Another method may be used to acquire an OCT tomographic image and motion contrast data. In the present embodiment, I(x, z) denotes an amplitude (of complex data after FFT processing) at a position (x, z) of tomographic image data I. M(x, z) denotes a motion contrast value at a position (x, z) of motion contrast data M.

Step S320

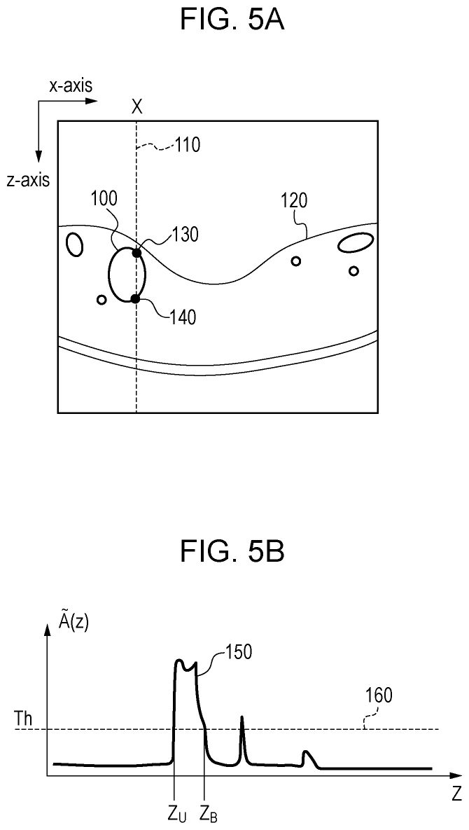

In step S320, the image feature acquisition unit 101-44, which is an example of a specification unit, specifies the position of an LVS in the Z direction. For this, the existence and position of an LVS with respect to the Z axis of the motion contrast data M(x, z) are specified using an unillustrated LVS specification unit in the image feature acquisition unit 101-44. In the present embodiment, the image processing unit 101-04 performs smoothing processing on the acquired motion contrast data M. As smoothing processing in this case, 2D Gaussian filter processing is performed on the entirety of an image and then moving average processing is performed for individual A-scans. Smoothing processing does not have to be limited to these types of processing. For example, other filters such as a moving median filter, a Savitzky-Golay filter, and a filter based on a Fourier transform may be used. In this case,
Ã(x, z)
   is a value obtained by performing smoothing processing on the motion contrast data M(x, z). FIG. 5A illustrates an example of
Ã(x, z)
   , which is a value obtained by performing smoothing processing on the motion contrast data M(x, z). FIG. 5A illustrates an example of a blood vessel structure 100 in an A-scan 110 performed for a retina 120. The size of the blood vessel structure in the Z direction is defined by the distance between an upper edge Z_{U} 130 and a lower edge Z_{B} 140. FIG. 5B is a profile plot of the A-scan 110. The lower edge ZB and the upper edge Z_{U} are determined on the basis of a threshold Th. If Z_{B}−Z_{U}>LVSs, it is determined that the blood vessel structure 100 is an LVS. Note that LVSs is a minimum blood vessel structure size determined to be an LVS. The lower edge Z_{B} and the upper edge Z_{U} are determined by positions where the profile plot 150 crosses the threshold 160. In the following description,
Ã(x, z)
   is a value obtained by performing smoothing processing on the motion contrast data M(x, z). In the present embodiment, empirically Th=0.1, and LVSs=0.018×Zmax. Note that Zmax is an A-scan size of the motion contrast data. Note that Th and LVSs are not limited to these values in the present embodiment, and Th and LVSs may be determined on the basis of, for example, optical properties of a tomographic imaging device (optical resolution and digital resolution, a scan size, density, and so on) or a signal processing method used to obtain motion contrast.

Step S330

In step S330, the image feature acquisition unit 101-44 confirms the occurrence of a projection artifact (PA) under the LVS on the basis of an OCT tomographic image, which is an example of OCT intensity information. That is, the unillustrated LVS specification unit in the image feature acquisition unit 101-44 confirms whether an intensity value I(x, z) of a tomographic image under the LVS has reduced. For example, the image processing unit 101-04, which is an example of the determination unit, determines whether to execute correction processing, which will be described later, for the blood vessel structure, by using information on a comparison result between OCT intensity information on the inside of the blood vessel structure (for example, Z_{U}<z≤Z_{B}) and OCT intensity information on the outside of the blood vessel structure (for example, Z>Z_{B}: a position deeper than that of the blood vessel structure). In this case, in a case where the OCT intensity information on the outside of the blood vessel structure is lower than the OCT intensity information on the inside of the blood vessel structure, the determination unit determines that the correction processing, which will be described later, is to be executed for the blood vessel structure. Depending on a blood vessel structure, no projection artifact may occur. When existing correction processing is performed for such a blood vessel structure similarly to as in the case where a projection artifact has occurred, an erroneous image may be generated. Thus, whether a projection artifact has occurred can be checked through a determination made by the determination unit described above. This can effectively reduce a projection artifact in motion contrast data. In this case, the information on the comparison result may be any information that enables the comparison result to be recognized. Note that, regarding a plurality of blood vessel structures of the subject's eye, whether to execute the correction processing, which will be described later, may be determined for each blood vessel structure. Consequently, it is possible to check whether a projection artifact has occurred for each of the plurality of blood vessel structures. This check processing is performed using the following Equation (1).

$$S = \{ \begin{matrix}1 & if MAX \{ I ( {z}_{U} < z \leq {z}_{B} ) \} > MAX \{ I ( z > {z}_{B} ) \} \\ 0 & otherwise\end{matrix} \qquad ( 1 )$$

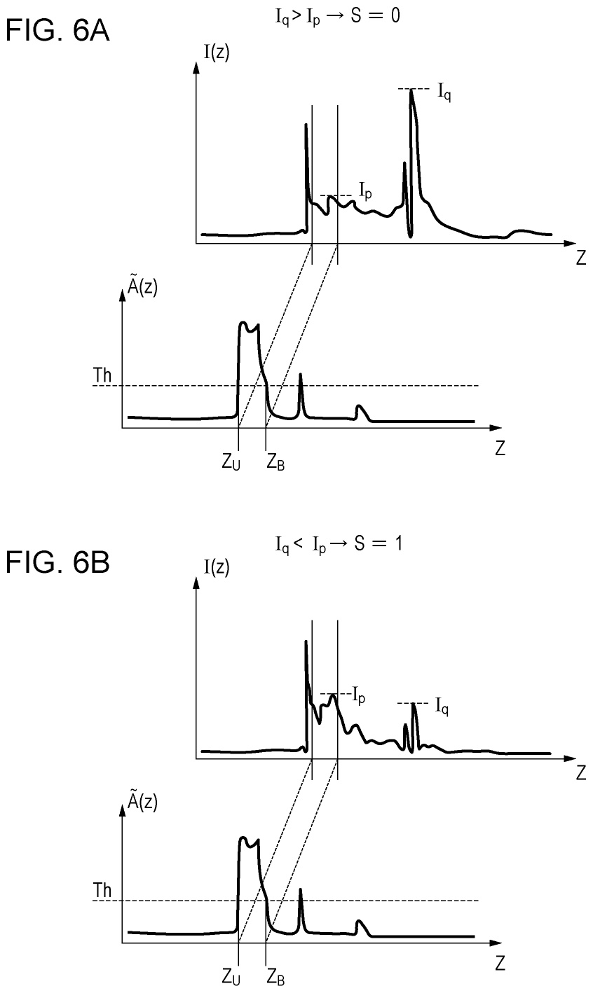

FIG. 6A illustrates a profile plot I(z) of a tomographic image I(x, z), and 6 B illustrates a profile plot
Ã(z)
   of
Ã(x, z)
   , which represents a value obtained by performing smoothing processing on motion contrast data M(x, z). FIGS. 6A and 6B respectively illustrate an example of a processing result when S=0 and an example of a processing result when S=1. In this case, the display controller 101-05 may cause the display unit 104 to display information on a determination result as to whether to execute this correction processing. Note that the information indicating the determination result may be any information with which the determination result is recognizable. In addition, on a blood vessel structure basis, the determination result may be changed (manually corrected) in accordance with a command issued on a display screen of the display unit 104 by the examiner. That is, in accordance with a command from the examiner, the state can be made to return to the state before the correction processing or the correction processing can be executed on a blood vessel structure basis.

Step S340

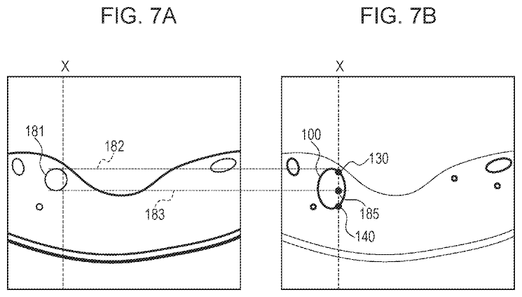

In step S340, the correction unit 101-43, which is an example of a size correction unit, corrects the size of the LVS. In a case where there is a highly reflective tissue near a blood vessel structure, the blood vessel becomes longer than it really is in the form of motion contrast data because of the highly reflective tissue. FIGS. 7A and 7B illustrate an example of such a phenomenon. FIG. 7A illustrates a tomographic image I(x, z). FIG. 7B illustrates the motion contrast image M(x, z). A dotted line 182 in FIGS. 7A and 7B indicates an upper edge of a blood vessel 181 illustrated in FIG. 7A and the position of the corresponding upper edge of the blood vessel structure 100 in FIG. 7B. A dotted line 183 in FIGS. 7A and 7B indicates a lower edge of the blood vessel 181 illustrated in FIG. 7A and the position of the corresponding portion of the blood vessel structure 100 in FIG. 7B. A position Z_{B} 140 illustrates a position shifted from the lower edge of the blood vessel. By correcting the position Z_{B} 140 in accordance with Equation (2), a blood vessel lower edge Z_{CB} 185 after the correction is obtained. That is, the size correction unit corrects the size of the blood vessel structure in the direction of depth of the subject's eye such that the size of the blood vessel structure is reduced. As a result, the position of the blood vessel structure can be specified with high accuracy. Note that, K is empirically set to 0.5 in the present embodiment; however, K may be set to any value other than zero.

Z_{CB}=Z_{B}−κ(Z_{B}−Z_{U}) (2)

Step S350

In step S350, the correction unit 101 43, which is an example of the calculation unit, calculates an attenuation coefficient γ(x, z) for the PA of the motion contrast. That is, using an unillustrated attenuation coefficient calculation unit in the correction unit 101-43, the attenuation coefficient γ(x, z) is calculated on the basis of 1. LVS information and 2. intensity value information (OCT intensity information) on the OCT tomographic image. For example, the calculation unit calculates the attenuation coefficient using OCT intensity information obtained at the position of a portion deeper than the blood vessel structure. As a result, an actual degree of effect of the projection artifact caused by the blood vessel structure can be reflected on the attenuation coefficient. Thus, for example, the correction processing can be prevented from being executed too severely or too lightly. That is, the projection artifact in motion contrast data can be effectively reduced. Note that the LVS information is an example of information on the position of the blood vessel structure. In this case, the information on the position may be any information that enables the position to be recognized, and may be, for example, a coordinate value in the direction of depth of the subject's eye (the Z direction) or may also be three-dimensional coordinate values. In addition, the information on the position is, for example, information on the distance from the blood vessel structure in the direction of depth of the subject's eye. In this case, the information on the distance may be any information that enables the distance to be recognized, and may be, for example, a numerical value with units or may also be something that can eventually lead to the distance such as two coordinate values. Next, details of the step S350 will be described.

Step S350A: A base attenuation coefficient γp(x, z) is calculated using Equation (3) on the basis of the PA confirmation occurrence S under the LVS, which is calculated in step S330, and the LVS information corrected in step S340.

$$γ p ( x , z ) = \{ \begin{matrix}{γ}_{0} + S ( x ) \cdot Δ C \cdot ( z - {z}_{CB} ( x ) ) & if ( z \geq {z}_{CB} ( x ) ) \\ {γ}_{0} & otherwise\end{matrix} \qquad ( 3 )$$

Note that γ0 is a fixed value, and empirically γ0=6 in the present embodiment. ΔC denotes attenuation of intensity of the PA under the LVS, and empirically ΔC=0.08 in the present embodiment. In order to avoid setting an extreme attenuation coefficient value, the upper limit of γp(x, z) is set to ymax. In the present embodiment, γmax=3.5. In the present embodiment, γp(x, z) defined by Equation (3) is a linear function with respect to a position Z; however, γp(x, z) may be a nonlinear function such as a power function or a rational function with respect to a position z.

Step S350B: In this step, using Equation (4), γc(x, z) is calculated by correcting γp(x, z) on the basis of the intensity value I(x, z) of the OCT tomographic image.

$${γ}_{C} ( x , z ) = \{ \begin{matrix}\frac{γ p ( x , z )}{{I}_{N} ( x , z )} & if ( z \geq {z}_{CB} ( x ) ) and S ( x ) = 1 \\ \frac{γ p ( x , z )}{{( {I}_{N} ( x , z ) )}^{2}} & otherwise\end{matrix} \qquad ( 4 )$$

Note that I_{N}(x, z) is a former OCT tomographic image I(x, z) which is normalized. I_{N}(x, z) is calculated as in the following. First, I(x, z) is smoothed by a 2D Gaussian filter. Then, each A-scan I(z) is smoothed using a moving average, and the smoothed A-scan I(z) is obtained. Next, each A-scan I(z) is independently normalized. Note that, in this case, normalization to 98% of the value of I(z) is performed. In the present embodiment, 98% is used, which is empirically determined. Note that, in the present embodiment, smoothing is not limited to the one using the smoothing function used in the above-described processing. For example, a moving median, a Savitzky-Golay filter, a Fourier transform based filter, or a combination of some of them may also be used. In this case, on a blood vessel structure basis, the calculated attenuation coefficient may be changed (manually corrected) in accordance with a command from the examiner. In addition, on a position basis in the direction of depth, the calculated attenuation coefficient may be changed (manually corrected) in accordance with a command from the examiner. Note that these commands from the examiner are, for example, commands issued on the display screen of the display unit 104 where information indicating the calculated attenuation coefficient is displayed. In addition, manual correction performed on the attenuation coefficient for a deep portion of a predetermined blood vessel structure may be reflected on the attenuation coefficient for a deep portion of another blood vessel structure. For example, the same amount of change as the amount of change of the attenuation coefficient for a deep portion of a predetermined blood vessel structure may be reflected on the attenuation coefficient for a deep portion of another blood vessel structure.

Step S360

In step S360, the correction unit 101-43 executes correction processing on the motion contrast data. Using Equation (5), the correction unit 101-43 calculates corrected information M_{COR}(x, z) by using the attenuation coefficient γc(x, z) with the original motion contrast data M(x, z).

$${M}_{cor} ( x , z ) = \{ \begin{matrix}M ( x , z ) & if z = 0 \\ M ( x , z ) \cdot {e}^{- \frac{{\sum }_{i = 0}^{i = z - 1} {M}_{cor} ( x , i )}{{γ}_{c} ( x , z )}} & otherwise\end{matrix} \qquad ( 5 )$$

Note that

$${\sum }_{i = 0}^{i = z - 1} {M}_{c o r} ( x , i )$$

is an accumulation of filtered motion contrast values from position 0 to z, where z is a z-domain coefficient. Note that correction of the motion contrast data may be correction of the entire motion contrast data, the correction may be performed in units of B-scan, or a depth range selected to generate a motion contrast en-face image may be corrected.

Step S370

In step S370, the projection unit 101-45 generates a motion contrast en-face image. The projection unit 101-45 projects the motion contrast image having the depth range based on the position of the layer boundary acquired by the image feature acquisition unit 101-44, and generates a motion contrast en-face image. An image having an arbitrary depth range may be projected; however, in the present embodiment, three types of motion contrast en-face image are generated for depth ranges that are a deep layer of the retina, an outer layer of the retina, and the choriocapillaris layer. As a projection method, either maximum intensity projection (MIP) or average intensity projection (AIP) may be selected, and projection is performed using MIP in the present embodiment.

Step S380

In step S380, the display controller 101-05 displays the motion contrast en-face images generated in step S370 on the display unit 104.

Step S390

In step S390, the image processing apparatus 101 associates the examination date and time and information used to identify the subject's eye with a group of acquired images (the SLO and tomographic images), imaging condition data of the group of images, the generated three-dimensional motion contrast image and motion contrast en-face images or corrected motion contrast data, and the associated generation condition data, and stores the associated data in the storage unit 101-02 and the external storage unit 102.

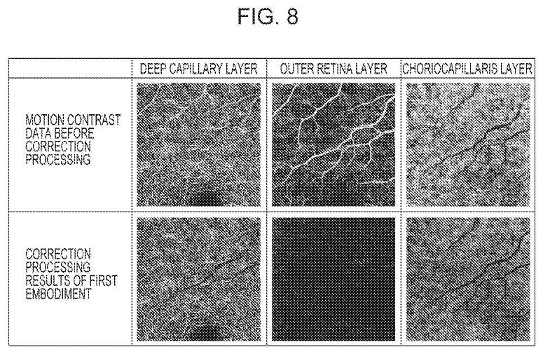

The description of the procedure for processing performed by the image processing apparatus 101 in the present embodiment is completed. FIG. 8 illustrates an example of displayed results. Note that the display controller 101-05 may align and display, on the display unit 104, a plurality of motion contrast en-face images that have undergone correction processing. In addition, the display controller 101-05 may display, on the display unit 104, one of the plurality of motion contrast en-face images that have undergone correction processing by performing switching therebetween in accordance with a selection made by the examiner (for example, a selection from the depth ranges such as a selection from the layers). In addition, the display controller 101-05 may display, on the display unit 104, at least one of the motion contrast en-face images that have not yet undergone correction processing and at least one of the motion contrast en-face images that have undergone correction processing by performing switching therebetween in accordance with a command from the examiner. In addition, the display controller 101-05 may display, on the display unit 104, a three-dimensional motion contrast image that has undergone correction processing.

Next, using FIG. 3B, specific processing steps for acquiring a tomographic image and motion contrast data, which is a fundus blood vessel image, in step S310 of the present embodiment will be described. Note that, for example, step S311 for setting the imaging conditions is an inessential step, and may thus be omitted in the present invention.

Step S311

In step S311, through an operation performed by the operator using the input unit 103, the imaging controller 101-03 sets OCTA-image imaging conditions to be set in the tomographic imaging device 100. Specifically, step S311 includes the following steps.
1) Select or register an examination set.
2) Select or add a scan mode for the selected examination set.
3) Set imaging parameters corresponding to the scan mode.

In addition, in the present embodiment, settings are set as in the following, and OCTA imaging is repeatedly performed (under the same imaging conditions) a predetermined number of times with short intermissions as appropriate in S312.
1) Register Macular Disease examination set.
2) Select OCTA-scan mode.
3) Set the following imaging parameters.
3-1) Scan pattern: Small Square
3-2) Scan region size: 3×3 mm
3-3) Main scan direction: horizontal direction
3-4) Scan spacing: 0.01 mm
3-5) Fixation lamp position: the fovea centralis
3-6) The number of B-scans per cluster: 4
3-7) Coherence gate position: vitreous body side
3-8) Default display report type: Single examination report

Note that the examination set indicates imaging steps (including scan modes) set for individual examination objectives and default display methods for OCT images and OCTA images acquired in individual scan modes. Based on this, an examination set which includes OCTA-scan mode in which settings for patients with macular diseases are set is registered under the name “Macular Disease”. The registered examination set is stored in the external storage unit 102.

Step S312

In step S312, upon acquiring an imaging start command from the operator, the input unit 103 starts repetitive OCTA imaging under the imaging conditions specified in 5311. The imaging controller 101-03 commands the tomographic imaging device 100 to execute repetitive OCTA imaging on the basis of the settings specified by the operator in 5301. The tomographic imaging device 100 acquires a corresponding OCT interference spectrum signal S(x, λ), and acquires a tomographic image on the basis of the interference spectrum signal S(x, λ). Note that the number of repetitive imaging sessions in this step is three in the present embodiment. The number of repetitive imaging sessions is not limited to three, and may be set to any arbitrary number. In addition, the present invention is not limited to cases where the imaging time intervals between the repetitive imaging sessions are longer than the imaging time intervals between tomographic image capturing sessions in each imaging session. Cases where the imaging time intervals between the repetitive imaging sessions are substantially the same as the imaging time intervals between tomographic image capturing sessions in each imaging session also fall within the present invention. In addition, the tomographic imaging device 100 also captures SLO images, and executes tracking processing based on an SLO moving image. In the present embodiment, a reference SLO image used in tracking processing in the repetitive OCTA imaging is a reference SLO image set in the first imaging session in the repetitive OCTA imaging, and the same reference SLO image is used in all the sessions in the repetitive OCTA imaging. Moreover, in addition to the imaging conditions set in S301, the same setting values are also used (are not changed) as to
Selection of the left or right eye
Whether to execute tracking processing during the repetitive OCTA imaging.

Step S313

In step S313, the image acquisition unit 101-01 and the image processing unit 101-04 generate motion contrast data on the basis of the OCT tomographic image acquired in S312. First, a tomographic image generation unit 101-11 generates tomographic images for one cluster by performing wave number conversion, a fast Fourier transform (FFT), and absolute value conversion (amplitude acquisition) on a coherent signal acquired by the image acquisition unit 101-01. Next, the position alignment unit 101-41 aligns the positions of the tomographic images belonging to the same cluster with each other, and performs overlay processing. The image feature acquisition unit 101-44 acquires layer boundary data from the overlaid tomographic image. In the present embodiment, a variable shape model is used as the layer boundary acquisition method; however, an arbitrary, known layer boundary acquisition method may be used. Note that layer boundary acquisition processing is inessential. For example, in a case where motion contrast images are generated only three-dimensionally and no two-dimensional motion contrast image projected in the depth direction is generated, layer boundary acquisition processing can be omitted. The motion contrast data generation unit 101-12 calculates motion contrast between adjacent tomographic images in the same cluster. As motion contrast, a decorrelation value M(x, z) is calculated on the basis of the following Equation (6) in the present embodiment.

$$M ( x , z ) = 1 - 2 \times \frac{A ( x , z ) \times B ( x , z )}{{A ( x , z )}^{2} + {B ( x , z )}^{2}} \qquad ( 6 )$$

In this case, A(x, z) denotes the amplitude (of complex data after FFT processing) at a position (x, z) of tomographic image data A, and B(x, z) denotes the amplitude at the same position (x, z) of tomographic data B. For M(x, z), 0≤M(x, z)≤1 is satisfied. As the difference between the two amplitudes increases, the value of M(x, z) approaches 1. Decorrelation arithmetic processing as in Equation (6) is performed on arbitrary, adjacent tomographic images (belonging to the same cluster), and an image having pixel values each of which is the average of (the number of tomographic images per cluster—1) motion contrast values obtained is generated as a final motion contrast image.

Note that, in this case, the motion contrast is calculated on the basis of the amplitudes of the complex data after FFT processing; however, the motion contrast calculation method is not limited to the above-described method. For example, motion contrast may be calculated on the basis of phase information on the complex data, or motion contrast may be calculated on the basis of both the amplitude information and the phase information. Alternatively, motion contrast may be calculated on the basis of the real part and the imaginary part of the complex data. In addition, decorrelation values are calculated as motion contrast in the present embodiment; however, the motion contrast calculation method is not limited to this. For example, motion contrast may be calculated on the basis of the difference between two values, or motion contrast may be calculated on the basis of the ratio between two values. Furthermore, in the description above, the final motion contrast image is obtained by obtaining the average of a plurality of acquired decorrelation values; however, the method for obtaining a final motion contrast image is not limited to this in the present invention. For example, an image having, as a pixel value, the mean value of or a maximum value out of the plurality of acquired decorrelation values may be generated as a final motion contrast image.

Step S314

In step S314, the image processing apparatus 101 associates the examination date and time and information used to identify the subject's eye with the group of acquired images (the SLO and tomographic images), imaging condition data of the group of images, the generated three-dimensional motion contrast image and motion contrast en-face images, and the associated generation condition data, and stores the associated data in the storage unit 10-02.

The steps described above are performed, and the description of the steps of processing for acquiring a tomographic image and motion contrast data of the present embodiment will be completed. With the above-described configuration, the effect of a projection artifact can be effectively reduced from an OCT tomographic image and motion contrast data by correcting the motion contrast data on the basis of the position of an LVS of an object to be imaged and intensity information on the OCT tomographic image.

### Second Embodiment

The first embodiment describes the method for reducing the effect of a projection artifact on the basis of the position of an LVS and OCT tomographic image intensity information and correcting motion contrast data. However, a projection artifact may be caused also in a small vessel structure and in a narrow blood vessel extending in the z direction. The present embodiment describes an example of a method for reducing the effect of a projection artifact in a small blood vessel structure and a narrow blood vessel extending in the z direction. The configuration of an image processing apparatus according to the present embodiment is the same as that of the first embodiment, and thus the description thereof will be omitted. Furthermore, a flow chart illustrating the process of operation processing of the entire system including the image processing apparatus of the present embodiment is the same as that of the first embodiment, and thus the description thereof will be omitted. Note that the following Equation (7) is used instead of γp(x, z) of Equation (3) used in step S350A.

$$γ p ( x , z ) = \{ \begin{matrix}{γ}_{0} + S ( x ) \cdot Δ C \cdot ( z - {z}_{CB} ( x ) ) + Δ A \cdot ( z - {z}_{CB} ( x ) ) & if ( z \geq {z}_{CB} ( x ) ) \\ {γ}_{0} & otherwise\end{matrix} \qquad ( 7 )$$

Note that ΔA is a term contributing to attenuation of a projection artifact in cases other than cases of LVSs (that is S(x)=0). In the present embodiment, ΔA=0.01, and ΔC=0.07; however, other values that are empirically determined may be used. With the above-described configuration, the effect of projection artifacts caused by an LVS and a narrow blood vessel can be effectively reduced from an OCT tomographic image and motion contrast data.

### Third Embodiment

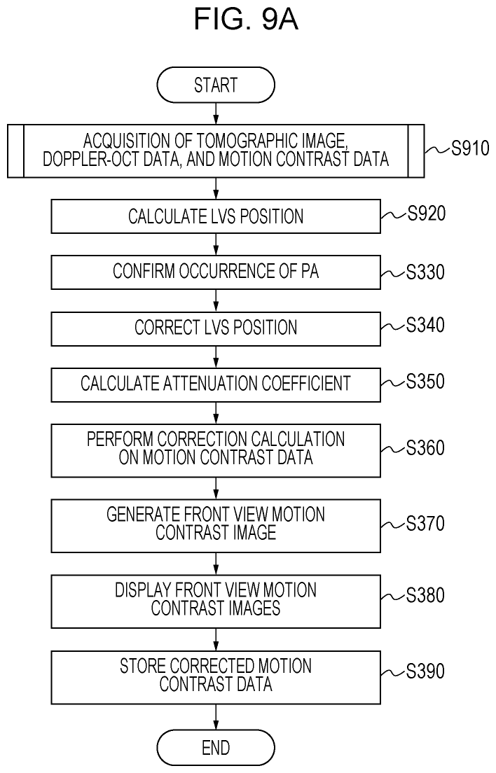

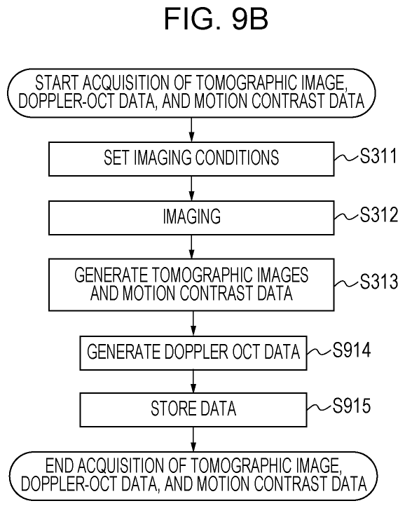

In the first embodiment, the method for specifying the position of an LVS on the basis of motion contrast data is described. The present embodiment describes another method for specifying the position of an LVS. The configuration of an image processing apparatus according to the present embodiment is the same as that of the first embodiment, and thus the description thereof will be omitted. Next, the procedure for operation processing of the entire system including the image processing apparatus of the present embodiment will be described using a flow chart illustrated in FIG. 9A. Note that steps S330 to S390 are the same as those of the flow chart in the first embodiment illustrated in FIG. 3B, and thus the description thereof will be omitted.

Step S910

In step S910, the image processing unit 101-04 acquires an OCT tomographic image, motion contrast data, and Doppler-OCT data. The image processing unit 101-04 may acquire an OCT tomographic image, motion contrast data, and Doppler-OCT data that have already been stored in the external storage unit 102; however, the present embodiment describes an example in which an OCT tomographic image, motion contrast data, and Doppler-OCT data are acquired by controlling the optical measurement system 100-1. Details of these processes will be described later. In the present embodiment, the way in which an OCT tomographic image, motion contrast data, and Doppler-OCT data are acquired is not limited to this acquisition method. Another method may be alternatively used to acquire a tomographic image, motion contrast data, and Doppler-OCT data. In the present embodiment, I(x, z) denotes an amplitude (of complex data after FFT processing) at a position (x, z) of tomographic image data I. M(x, z) denotes a motion contrast value at a position (x, z) in motion contrast data M. D(x, z) denotes a Doppler value at a position (x, z) of Doppler-OCT tomographic image data corresponding to the tomographic image data I.

Step S920

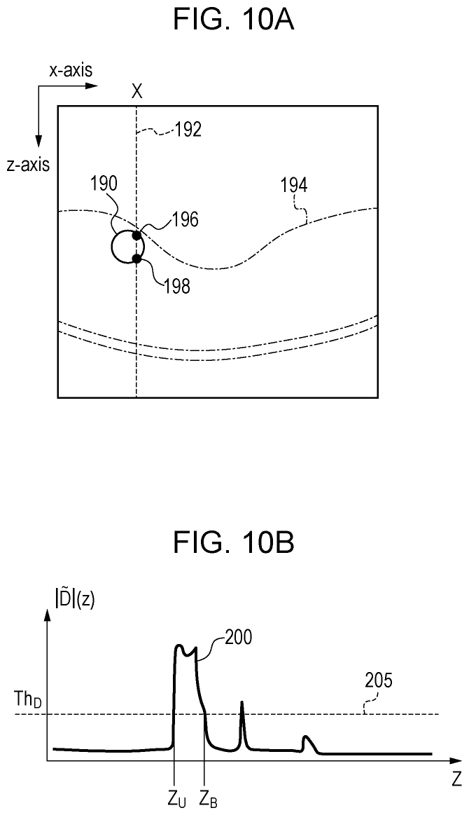

In step S920, the image feature acquisition unit 101-44 specifies the position of an LVS in the Z direction. Thus, the existence and position of an LVS with respect to the Z axis of the Doppler-OCT data D(x, z) are specified using the unillustrated LVS specification unit in the image feature acquisition unit 101-44. Details of the processing will be described using FIGS. 10A and 10B. In the present embodiment, first, smoothing processing is performed on acquired Doppler-OCT data D. As smoothing processing in this case, 2D Gaussian filter processing is performed on the entirety of an image and then moving average processing is performed for individual A-scans. Smoothing processing does not have to be limited to these types of processing. For example, other filters such as a moving median filter, a Savitzky-Golay filter, and a filter based on a Fourier transform may be used. FIG. 10A illustrates an example of smoothed Doppler-OCT data, which is
   |\tilde{D}|(x, z).
   FIG. 10A illustrates an example of a blood vessel structure 190 in a Doppler A-scan 192 performed for a retina 194. The size of the blood vessel structure 190 in the Z direction is defined by the distance between an upper edge Z_{U} 196 and a lower edge Z_{B} 198. FIG. 10B illustrates a profile plot of the Doppler A-scan 192. The lower edge Z_{B} and the upper edge Z_{U} are determined on the basis of a threshold Th_{D}. If Z_{B}−Z_{U}>LVSds, it is determined that the blood vessel structure 190 is an LVS. Note that LVSds is a minimum blood vessel structure size determined to be an LVS. The lower edge Z_{B} and the upper edge Z_{U} are determined by positions where the profile plot 200 crosses the threshold Th_{D}. In the present embodiment, empirically Th_{D}=0.3π, and LVSds=0.018×Zmax. Note that Zmax is an A-scan size for motion contrast data. Note that Th_{D} and LVSds are not limited to these values in the present embodiment, and Th_{D} and LVSds may be determined on the basis of, for example, optical properties of a tomographic imaging device (optical resolution and digital resolution, a scan size, density, and so on) or a signal processing method used to obtain motion contrast. In the present embodiment, the position of Z_{B} does not have to be corrected, and thus hereinafter Z_{CB}=Z_{B}.

The description of the procedure for the motion contrast data PA correction processing performed by the image processing apparatus of the present embodiment is completed.

Next, using FIG. 9B, specific processing steps for acquiring the tomographic image, motion contrast data, which is a fundus blood vessel image, and Doppler-OCT data in step S910 in the present embodiment will be described. Note that steps S311 to S313 are the same as those of the flow chart in the first embodiment illustrated in FIG. 3B, and thus the description thereof will be omitted.

Step S914

In step S914, using Equation (8), the image acquisition unit 101-01 and the image processing unit 101-04 generate D(z) in a Doppler-OCT data A-scan x on the basis of an OCT interference spectrum signal S(x, j, λ) acquired in S312. In this case, j=1, . . . , r. Note that r denotes an oversampled spectrum, and r=2 in the present embodiment.

$$D ( z ) = t a {n}^{- 1} [ \frac{1}{r} {\sum }_{j = 1}^{r} [ s ( j , z ) \cdot {s}^{*} ( j + 1 , z ) ] ] \qquad ( 8 )$$

Note that a complex number S(j, z) is a Fourier transform result of the OCT interference spectrum signal S(x, j, λ). In addition, S*(j+1, z) is a complex conjugate of S(j+1, z). Note that the method for generating Doppler-OCT data D(z) in the present embodiment does not have to be the one based on the above-described equation. For example, the phase shift Doppler method, the Hilbert transform phase shift Doppler method, or the STdOCT method may be used.

Step S915

In step S915, the image processing apparatus 101 associates the examination date and time and information used to identify the subject's eye with a group of acquired images (the SLO and tomographic images), imaging condition data of the group of images, the generated three-dimensional motion contrast image and motion contrast en-face images, the Doppler-OCT data, and the associated generation condition data, and stores the associated data in the storage unit 101-02.

The steps described above are performed, and the description of the steps of processing for acquiring a tomographic image, motion contrast data, and Doppler-OCT data is completed in the present embodiment.

With the above-described configuration, the effect of a projection artifact can be effectively reduced from an OCT tomographic image, motion contrast data, and Doppler-OCT data on the basis of the position of an LVS of an object to be imaged and intensity information on the OCT tomographic image.

### Fourth Embodiment

In the first embodiment, the method for reducing the effect of a projection artifact on the basis of the position of an LVS and OCT tomographic image intensity information and correcting motion contrast data is described. In the present embodiment, an attenuation coefficient calculation method will be described by further considering features of anatomical tissue that is an object to be imaged. The configuration of an image processing apparatus according to the present embodiment is the same as that of the first embodiment, and thus the description thereof will be omitted. Furthermore, a flow chart illustrating the procedure for operation processing of the entire system including the image processing apparatus of the present embodiment is the same as that of the first embodiment, and thus the description thereof will be omitted. Note that the following Equation (9) is used instead of γp(x, z) of Equation (3) used in step S350A.

γp=(x, y)=γ(x, z)*μ(x, z) (9)

Note that a function μ(x, z) depends on information on the layer boundary of the retina, and is defined by the following Equation (10). Note that the information on the layer boundary is acquired using the image processing unit 101-04, which is an example of an analysis unit, by analyzing OCT intensity information. In this case, the information on the layer boundary may be any information that enables, for example, the type and position of the layer boundary to be recognized.

$$μ ( x , z ) - \{ \begin{matrix}\frac{γ 0 ( x , z )}{γ ( x , z )} & if z > {z}_{RPE} ( x ) \\ 1 & otherwise\end{matrix} \qquad ( 10 )$$

In this case, Z_{RPE}(x) denotes a position Z of the retinal pigment epithelium (RPE) of the retina in an A-scan X. Moreover, γ(x, z) is as follows.

$$γ ( x , z ) = \{ \begin{matrix}{γ}_{0} + S ( x ) \cdot Δ C \cdot ( z - {z}_{CB} ( x ) ) & if ( z \geq {z}_{CB} ( x ) ) \\ {γ}_{0} & otherwise\end{matrix} \qquad ( 11 )$$

Note that, in the present embodiment, μ(x, z) does not have to be based on the RPE, and may be based on, for example, another layer. With the above-described configuration, an attenuation coefficient can be calculated more accurately by considering the features of tissue that is an object to be imaged.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. An image processing apparatus for reducing a projection artifact in motion contrast data of a subject's eye, the image processing apparatus comprising:
a calculation unit configured to calculate, using information on a position of a blood vessel structure of the subject's eye and optical coherence tomography (OCT) intensity information on the subject's eye, an attenuation coefficient regarding attenuation of the motion contrast data in a direction of depth of the subject's eye; and
a correction unit configured to execute correction processing on the motion contrast data using the calculated attenuation coefficient.

2. The image processing apparatus according to claim 1, wherein the calculation unit calculates the attenuation coefficient using information on a distance from the blood vessel structure in the direction of depth and the OCT intensity information.

3. The image processing apparatus according to claim 1, wherein the calculation unit calculates the attenuation coefficient using OCT intensity information obtained at a position of a portion deeper than the blood vessel structure.

4. The image processing apparatus according to claim 1, further comprising:
a determination unit configured to determine, using information on a comparison result between OCT intensity information on an inside of the blood vessel structure and OCT intensity information on an outside of the blood vessel structure, whether to execute the correction processing for the blood vessel structure, wherein
the calculation unit calculates the attenuation coefficient in a case where it is determined that the correction processing is to be executed.

5. An image processing apparatus for reducing a projection artifact in motion contrast data of a subject's eye, the image processing apparatus comprising:
a correction unit configured to execute correction processing on the motion contrast data using an attenuation coefficient regarding attenuation of the motion contrast data in a direction of depth of the subject's eye; and
a determination unit configured to determine, using information on a comparison result between OCT intensity information on an inside of a blood vessel structure of the subject's eye and OCT intensity information on an outside of the blood vessel structure, whether to execute the correction processing for the blood vessel structure.

6. The image processing apparatus according to claim 4, wherein the determination unit determines that the correction processing is to be executed for the blood vessel structure in a case where the OCT intensity information on the outside of the blood vessel structure is lower than the OCT intensity information on the inside of the blood vessel structure.

7. The image processing apparatus according to claim 4, wherein regarding a plurality of blood vessel structures of the subject's eye, the determination unit determines whether to execute the correction processing for each blood vessel structure.

8. The image processing apparatus according to claim 4, further comprising: a display controller configured to cause a display unit to display information indicating a determination result as to whether to execute the correction processing.

9. The image processing apparatus according to claim 8, wherein it is possible to change, in accordance with a command on a display screen of the display unit from an examiner, the determination result as to whether to execute the correction processing.

10. The image processing apparatus according to claim 1, wherein it is possible to change the calculated attenuation coefficient in accordance with a command from an examiner.

11. The image processing apparatus according to claim 1, further comprising: a specification unit configured to specify the blood vessel structure using the motion contrast data.

12. The image processing apparatus according to claim 1, further comprising: a size correction unit configured to correct a size of the blood vessel structure such that the size of the blood vessel structure is reduced in the direction of depth of the subject's eye.

13. The image processing apparatus according to claim 1, further comprising: a specification unit configured to specify the blood vessel structure using Doppler-OCT data.

14. The image processing apparatus according to claim 1, further comprising: an analysis unit configured to acquire information on a layer boundary of the subject's eye by analyzing the OCT intensity information, wherein
the calculation unit calculates the attenuation coefficient using information on the position, the OCT intensity information, and information on the layer boundary.

15. The image processing apparatus according to claim 1, further comprising: an image processing unit configured to execute smoothing processing on the motion contrast data.

16. An image processing method for reducing a projection artifact in motion contrast data of a subject's eye, the image processing method comprising:
Calculating, using information on a position of a blood vessel structure of the subject's eye and OCT intensity information on the subject's eye, an attenuation coefficient regarding attenuation of the motion contrast data in a direction of depth of the subject's eye; and
executing correction processing on the motion contrast data using the calculated attenuation coefficient.

17. An image processing method for reducing a projection artifact in motion contrast data of a subject's eye, the image processing method comprising:
executing correction processing on the motion contrast data using an attenuation coefficient regarding attenuation of the motion contrast data in a direction of depth of the subject's eye; and
determining, using information on a comparison result between OCT intensity information on an inside of a blood vessel structure of the subject's eye and OCT intensity information on an outside of the blood vessel structure, whether to execute the correction processing for the blood vessel structure.

18. A non-transitory computer-readable storage medium storing a program for causing a computer to execute the image processing method according to claim 16.
